(19) United States
(12) Patent Application Publication
(10) Pub. No.: US 2023/0218490 A1
(43) Pub. Date: Jul. 13, 2023

(54) TRUFFLE CONFIGURED COSMETIC ARTICLE

(71) Applicant: REA Innovations, Inc., Bainbridge Island, WA (US)

(72) Inventor: Robb Akridge, Bainbridge Island, WA (US)

(21) Appl. No.: 18/180,739

(22) Filed: Mar. 8, 2023

Related U.S. Application Data
(63) Continuation of application No. 15/695,661, filed on Sep. 5, 2017.

(51) Int. Cl. A61K 8/11; B01F 31/44; B01F 27/1125; A61K 8/73; B01F 35/32; A61K 8/92; A61K 8/55; B01F 35/71; B01F 35/93; B01F 35/45; A45D 34/00; A45D 44/00; B01F 33/501; B01F 35/53; A61Q 19/08; B01F 27/90; B01F 31/441; A61Q 19/00; B01F 27/808; B01F 31/445; A45D 40/00; B01F 101/21; B01F 35/90

(52) U.S. Cl. CPC A61K 8/11; B01F 31/449; B01F 27/1125; A61K 8/732; B01F 35/32025; A61K 8/922; A61K 8/553; B01F 35/7131; B01F 35/93; B01F 35/451; A45D 34/00; A45D 44/00; B01F 33/5011; B01F 33/5014; B01F 35/53; A61Q 19/08; B01F 27/90; B01F 31/441; B01F 35/3202; A61K 8/92; A61Q 19/00; B01F 27/808; B01F 31/445; A45D 40/0087; B01F 2101/21; B01F 2035/99; A61K 2800/805; A61K 2800/87; A61K 2800/88

(57) ABSTRACT

A truffle configured cosmetic article which includes a protective outer shell, an inner volume of cosmetic and a capsule positioned in the cosmetic containing active ingredients. An appliance is adapted to receive the article, heat and blend the article to produce a warm skin formulation which can be applied by the user. The article is produced with the use of a mold. A combination of wax and coconut oil is melted and applied to most of the interior surface of the mold, defining a volume into which a cosmetic is applied as well as a capsule additive. The article is then sealed with additional wax and coconut oil material. The article is then released and ready for use in the appliance or to be wrapped.

### RELATED APPLICATIONS

The present application is continuation of U.S. patent application Ser. No. 15/695,661, which was filed on Sep. 5, 2017, the complete disclosure of which is hereby incorporated by reference.

### TECHNICAL FIELD

This invention relates generally to cosmetics and more particularly to individual cosmetic units or portions which are then blended prior to actual use.

### BACKGROUND OF THE INVENTION

Typical high end or signature cosmetic departments will display multiple brands, each with their own signature look and appeal. Each cosmetic brand typically has a glass counter, some with stools or other seating arrangements. On top of the retail counter, in glass cases and behind the counter typically are arranged bottles, tubes and jars according to the cosmetic purpose, including, for example, anti-aging moisturizers or skin conditioners, and several different cosmetic formulations. In each arrangement or brand, there are a large number of different containers; accordingly, there is typically a beauty advisor to assist and to sell to the individual customers. The beauty advisor will, after consulting with the customer, typically advise a skin care routine of three basic steps, to cleanse, treat and protect. For each step, there are numerous bottles and jars containing specialized formulations to be purchased. In many cases, a customer will leave with a large number of individual cosmetic items. The individual containers, each with their own box, are bagged and given to the customer. Typically, each container has a three month supply of cosmetics.

The contents of a given container are usually mixed and filled in a manufacturer's facility approximately 6-9 months prior to being distributed to retailers. It typically takes an additional three months to get the containers placed on a shelf in each store, and the products can then sit of the shelf for as long as 2 years until purchased. The products thus must be robust to maintain their effectiveness for such a long time. The long timeline is still undesirable, however, because of its effect on freshness, as well as the large number of individual containers typically purchased by the customer during a single consultation. All of the luxury brands of cosmetic operate in basically the same way and have therefore the same disadvantages.

The present invention is directed toward a significantly different approach to the distribution and sale of high end/luxury cosmetics.

### SUMMARY OF THE INVENTION

Accordingly, present invention includes: a cosmetic article comprising: a water resistant outer layer; and an inner volume of cosmetic material, wherein the cosmetic and the outer layer are blendable to produce a skin formulation for application by a user. The present invention also includes: a process for manufacturing a cosmetic article comprising the steps of: providing a mold for a cosmetic article/or articles; melting protective shell material and forming it on an interior surface of the mold; applying cosmetic material to an interior volume defined by the protective shell; sealing the cosmetic with additional shell material so that it is completely surrounded by the shell material, defining an outer layer for the article; and releasing the cosmetic article/or articles from the mold. The present invention further includes: an appliance for blending a cosmetic article having a protective outer layer and an inner volume of cosmetic cream, comprising: a container for the cosmetic article having a heating assembly and elements for blending the article into a skin formulation; a motorized base member to which the container is connectable for moving the blending elements; and a mechanism operable by the user for operating the base member to blend and heat the cosmetic article into a formulation.

### BRIEF DESCRIPTION OF THE DRAWINGS

FIG. 1A is an elevational view of a truffle-like cosmetic portion of the present invention, similar in size and shape to a confectionary truffle.

FIG. 1B is a cross sectional view of the cosmetic portion of FIG. 1A.

FIG. 2 is an elevational view of the truffle portion of FIG. 1 covered in a wrapper.

FIG. 3 is a schematic view of a plurality of truffle portions arranged in a truffle signature box.

FIG. 4A is a perspective view of an appliance for transforming the truffle cosmetic portion of FIG. 1 into a formulation which is applied by a user.

FIG. 4B is a perspective view of the appliance of FIG. 4A in an opened position, displaying the formulation for use.

FIG. 5A is a perspective view of a mold plate for producing the truffle portions of FIG. 1.

FIG. 5B is a cross sectional view showing a first step in the manufacture of the truffle portions.

FIG. 5C is a cross sectional view showing a second step in the manufacture of the truffle portions.

FIG. 5D is a third step in the manufacture of the truffle portions.

FIG. 5E is a fourth step in the manufacture of the truffle portions.

### BEST MODE FOR CARRYING OUT THE INVENTION

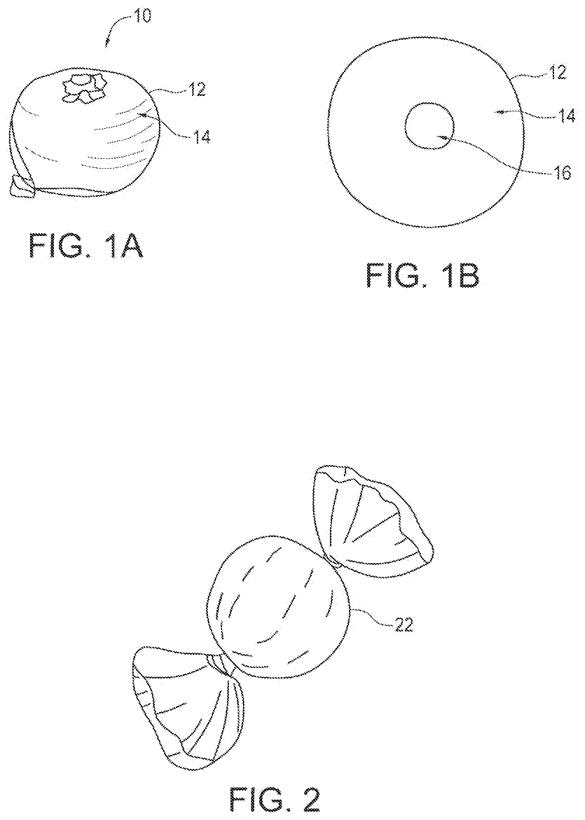

FIGS. 1A and 1B show the truffle cosmetic portion or member of the present invention. The word truffle used herein means a product portion or member described herein which is configured to have the appearance of a confectionary truffle. It is, however, not a confectionary product. The truffle shown generally at 10 includes a protective coat outer layer 12, an inner volume of cosmetic 14 and, in some embodiments, a central capsule 16, such as typically found in the nutrition industry, also with a protective coating. The outer protective coat acts as a water barrier, preventing water from escaping the cosmetic 14 and water from entering the truffle portion. The protective coat has a melting point typically in the range of 90-110 degrees Fahrenheit. A typical range of thickness is 0.5 mm-5 mm, with a volume of 0.5-2.0 ml or in some cases 4 ml or larger, although these dimensions can vary further. The outer layer is chemically compatible with the cosmetic 16 and the central capsule when one is present. The outer layer 12 comprises various materials including, but not limited to, materials found in enteric coatings, including fatty acids, plant and animal waxes (e.g. paraffin, carnuba wax, beeswax, stearic acid, lanolin, soy wax), shellac and animal fibers, fats (e.g. vegetable fats), resins, oils, starches, among others. Wax is an excellent barrier to water but is relatively brittle in its pure form so that a typical protective coat/outer layer will consist of a combination of wax and coconut oil in a 1:2 or 1:3 ratio. Resin (or similar material) can be mixed with the wax/oil combination to alter properties of the outer layer, typically resulting in a slightly higher melting point. Alternatives to coconut oil include coconut butter, argon Oil and Shea Butter. Still other possibilities include mineral oil and paraffin. In addition to a combination of wax and oils, other materials, including sodium alginate/calcium, agar, agarose, carrageenan, gelatin and hydra gels can be used, all of which are designed to protect the internal cosmetic material 14.

The inner volume of cosmetic material 14 can vary significantly. The cosmetic can be those used to cleanse, hydrate, treat, protect or even color the skin. The formula can be either aqueous or oil based. The cream could be pure Shea Butter (i.e. oil). It could also be a complex, special formula supplied by a contract manufacturer. However, it should melt in the range of 90-110 degrees Fahrenheit and be blendable with the other parts of the truffle including in particular the outer coating. The cosmetic could be various skin creams which treat oily skin, dry skin, acne skin, sensitive skin and normal skin. The function of the cream could be hydrating, or a barrier cream which prevents water loss from the skins' surface, or other treatment creams, including anti-aging creams, calming creams, and nourishing creams.

The center capsule 16 is similar to conventional capsules. This part of the truffle is not essential but is important in particular cases; the truffle can comprise the protective outer layer and the cosmetic alone. The capsule 16 when present will typically be a capsule made of gelatin or other encapsulating materials, including non-animal products such as cellulose, ranging in volume from 10 μl-500 μl, containing vitamins or an oil, or other active ingredients or a fragrance. Various beauty oils can be used including, but not limited to, for example, argan oil, mango oil, sandalwood, avocado oil, coconut, grape seed, and evening primrose. Essential oils can also be used for skincare, including for instance, rose oil, carrot seed, frankincense, geranium and many others, which are directed toward skin care issues, including acne, aging, scarring, oiliness, dryness or elasticity. The capsule could also include prescription ingredients. Some of the active ingredients could be vitamins A, E, C, BHA (beta-hydroxy acid), salicylic acid, AHA (alphahydroxy acid), various proteins and peptides, skin darkening or lightening agents and liposomes.

The capsule must not dissolve within the cream center prior to blending. Further, it should not be a solid. It must melt or dissolve when placed in an appliance for blending and heating. Soft gel caps can be used as well as the harder gelatin capsules or even wax capsules. The capsule ingredients are enclosed within the gel cap or similar substance to remain separate from the cream until the truffle is blended. Several capsules can be included in the center of the cosmetic volume 14 of a single truffle. In such an arrangement, a synergy could result when the truffle is blended and heated.

In general summary, the truffle member or portion includes an outer protective coating, typically a wax/oil combination, an inner cosmetic volume, and a capsule containing an active or other ingredient described above. The truffle can be decorated with various components, including mineral or metal elements, in various ways, including various design/initials.

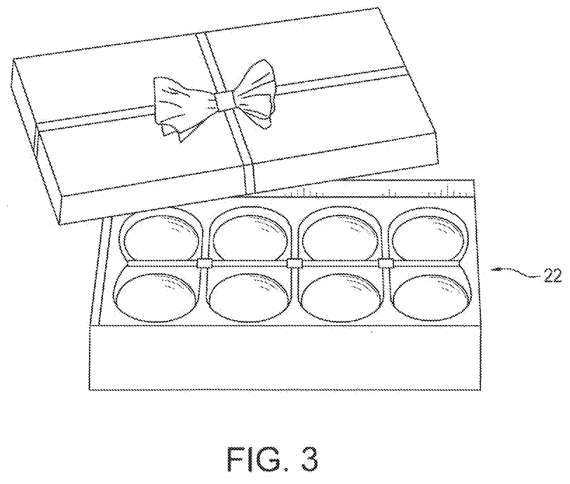

Each truffle cosmetic member can be individually wrapped at 20 to extend shelf life or to increase the attractiveness of the truffle, as illustrated in FIG. 2. The wrapper 20 can also have various designs and can be of various materials, including foil and paper, among others. The individuality of the truffle can be embellished by the wrapper.

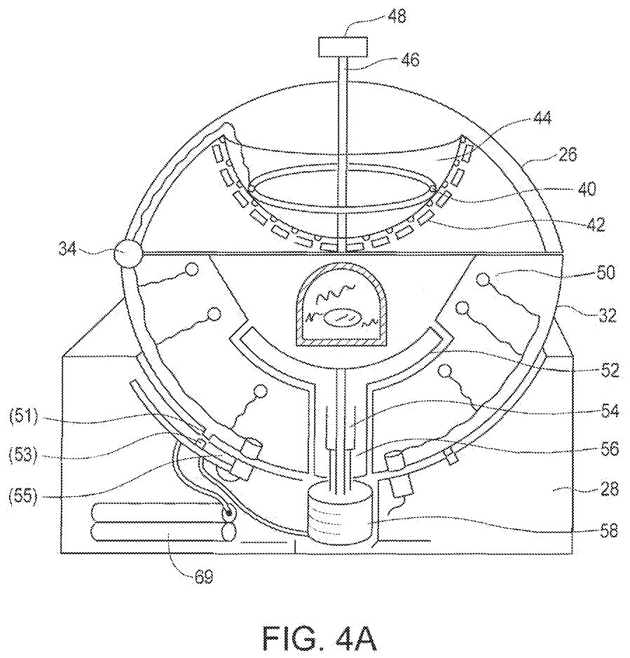

A plurality of individual truffles can be placed in a special truffle box 22 as shown in FIG. 3. The truffle box 22 has the appearance of an assortment of chocolate candy truffles, providing a distinctive brand appearance which is substantially different than a plurality of containers, jars, tubes etc. provided by existing luxury brands for its cosmetics. The truffle member concept of the present invention is thus substantially different not only in its structural approach to cosmetics and the conventional cosmetics counter but in the appearance and commercial presentation of the truffle member.

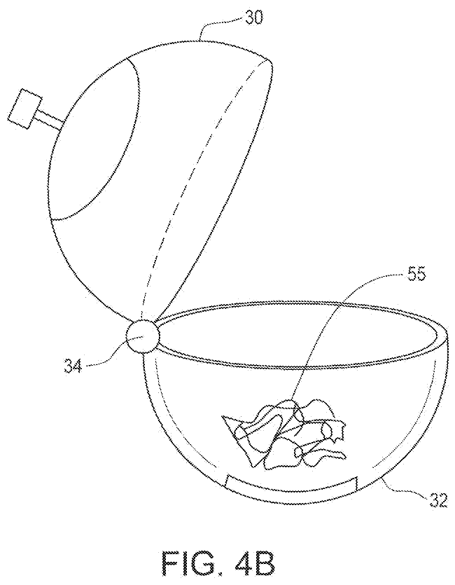

In order for the user to make use of the truffle cosmetic member described above, the truffle must be placed in an appliance, e.g. an amulet container which is designed to hold the truffle and operate on the truffle to make it usable. The amulet is typically spherical and is shown in cross sectional form in FIG. 4A. The amulet 26 is positioned on a motor platform 28 which can be positioned on a shelf or counter or the like. The appliance can be powered by batteries (direct current) or passively charged. The spherical amulet includes a top portion 30 which can be lifted off a lower portion 32 by a side hinge 34. The top portion in use is lifted up, and the truffle 36 is inserted into the lower portion after the wrapper, if present, is removed from the truffle cosmetic member. The top portion 30 includes a cup-like depending portion 44 which is attached to the inner surface of top portion 30. The depending cup portion 44 includes upper heating elements 40. A mesh grid 42 surrounds the depending portion 44. A plunger 46 extends down through dependent portion 44 with the lower end thereof secured to the mesh grid 42. The upper end of the plunger 46 extends out of the top portion, terminating in a push button element 48.

The lower part includes another heating element 50 and a rotating curved blade 52 which has a mechanical connection 54 which is mated to a drive member 56 from motor 58, with the motor being powered by a battery 69 which can be charged.

In operation, push button 48 is moved downwardly by the user, moving the mesh 42, mashing the truffle against blades 52. In addition to mashing the truffle member, pushing down on push button 48 causes the entire amulet to move downward, where a small projection 51 on the lower surface of the amulet actuates an on/off button 53. This starts a software program on circuit board 55 to actuate blades 52, which are retractable, and provides current to heating elements 40 in the top portion 50 and in the bottom portion of the amulet. The blades operate at a sufficient speed to blend the parts of truffle together, resulting in a creamy formulation. Although the described embodiment uses moving blades, with a revolving or an oscillating movement, other arrangements can be used to produce the blending, including for instance ultrasound or other mechanical means, such as a plunger The depending part 44 in the top portion is in the same shape as the mesh grid and is textured to mate with the mesh grid so that material is pushed out of the mesh in operation as the mesh grid is moved upwardly back against the dependent part 44. The truffle member is blended with heat, to form a conventional, usable formulation.

Referring to FIG. 4B, the formulation is accessible by lifting the top portion 30 of the amulet off the bottom portion 32 abut hinge 34. The size of the amulet in conjunction with the size of the truffle produces a desired supply of the formulation 55, i.e. a few days (or even one application) to perhaps a month supply. When the formulation is used up, the amulet can be washed to receive a new truffle. While an appliance is typically used to blend and warm the truffle into a formulation, in some cases, when the truffle has a particular composition, the blending and warming of the truffle can be accomplished by hand action alone.

The truffle of FIGS. 1A and 1B can be manufactured in various ways. One current method is described as follows. It should be understood that the various process steps described below are illustrative only and can be varied, depending upon the desired characteristics and function of the final blended formulation.

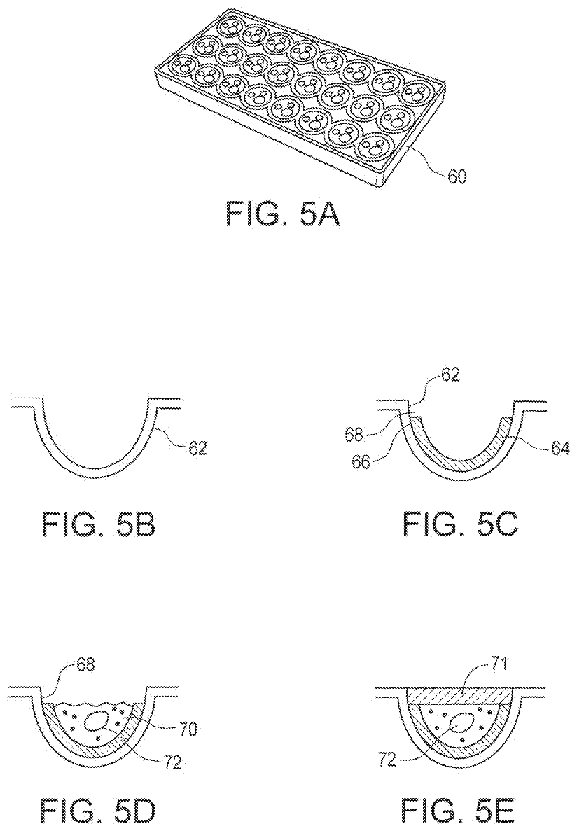

In the present arrangement, the truffle members are processed individually in a mold plate such as 60 shown in FIG. 5A. In a double boiler filled with water, the top part of the boiler also has water. The water in the top part is heated to simmering. 50 grams of a wax mixture is added to the upper part and slowly stirred until the wax is melted. The internal gel capsule is dipped into the melted wax, insuring that the capsule is completely coated, then placed on wax paper to cool. In another bowl is added wax, butter and oil (wax/butter/oil mixture at the desired ratio, typically in the range of 1:1:1-1:5:5). In some cases, pure butter (e.g. cocoa butter) could be used or wax butter. This bowl is placed in the heated double boiler and the mixture stirred until it is completely melted. Typically the temperature will depend on the selected ratio, but usually ranges from 90° F. to 110° F. This is the outer layer material of the truffle.

The inside of each truffle mold 62 FIG. 5B in the mold plate 22 is first coated with a releasing agent such as olive oil. 3 ml of the melted wax/oil mixture is poured into each mold. The mold can be warmed to slow the natural solidification of the wax/oil mixture. FIG. 5C shows mold 62 and a wax/oil shell 64. The mold is then placed in a refrigerator to cool for a few minutes. The mold is removed and inverted to remove any remaining liquid mixture. At this point the mold edge 66 has a slight head space 68 which is below the top of the mold. This headspace is filled with additional wax/oil mixture once the other components are added.

A pastry bag or similar container filled with the desired cosmetic 70, such as one of the various possibilities discussed above, is used to fill each shell with approximately 0.5-1.0 ml of cosmetic. This is shown in FIG. 5D. The cream should not extend above the mold shell. At this point, one or more wax coated capsules 72 can be inserted into the cosmetic, if desired.

The remaining head space is then filled with liquid wax/oil mixture. The cosmetic is completely covered by the wax/oil mixture, shown at 71 in FIG. 5E with the interior cosmetic 70 and the capsule 72.

The mold is then placed in a freezer for approximately 15 minutes to solidify the truffle member. The mold is then removed from the freezer, inverted and the truffle members released.

It should be understood that the manufacturing process disclosed above is one example only of how to make the truffle members.

Decoration of the resulting truffle is referred to briefly above. This can occur at several places during the process of manufacture. Edible gold flakes can be added to the releasing agent, such as olive oil, to give a golden sheen to the entire surface. Once the truffles are released from the mold, gold leaf 74 (FIG. 1A) can be added to the surface using a small brush. Decorations can be other elements including mica or other minerals such as silver, mineral make-up powder, even food grade dyes can be used to decorate and embellish the appearance of the truffle members.

As discussed above, the individual truffles are then made available for display, either wrapped or unwrapped, depending on the truffle context. The truffles are typically provided in a display counter at retail, similar to a display used in a high end chocolate shop. A beauty advisor is typically needed to help select different truffles for each customer's skin type and need. Consumers purchase their two week or typically one month supply of each truffle. Generally, a single truffle will be for one day or two weeks or one month, although this could vary. As one example, for anti-aging, the beauty advisor could recommend truffles with Retinol capsules, where each month's supply would be an anti-aging truffle with Retinol treatment capsules. The first month's supply (30 truffles) could include a shea cream cosmetic center while the next month a proprietary cosmetic could be included and the following month a coconut butter cosmetic. The same approach could be used with different fragrances. Further, there may be different truffle products for morning and for at night.

At this point, once the truffle selection has been made, the beauty advisor will place each truffle in a segmented specialty box. The box is wrapped and the consumer carries it away like a chocolate box or a hat box. The size of the box will vary depending on the need or the skin issue of the customer.

First time customers will have to purchase the appliance, i.e. the amulet and the motor mount. The beauty advisor will demonstrate the use of the appliance with a truffle. The truffle is first placed in the amulet. The amulet is then placed on the motor mount. Operating the pushbutton will produce the predetermined mashing, blending, and heating of the truffle. When completed, the customer can remove the amulet. The amulet is then opened, typically revealing a warm, fragrant, fluffy cream that can be applied to the skin directly by the user. Typically, the motor mount will only be needed to blend the truffle one time. However, the amulet can be used to heat the blended formulation for each actual use if desired. The customer can use the appliance at home.

There are several advantages of the present invention over existing luxury cosmetic brands, including fresh preparation, customized for the individual skin and easier to use. The resulting warm formulation is easy to apply and absorbs better into the skin, providing a novel sensory experience. It should be understood that each truffle member is a single use to avoid spoilage, as can occur with a conventional three month bottle supply of cosmetic. It should also be understood that the size of the truffle can vary depending on the need to create up to a month's supply in the amulet.

As indicated above, the amulet is reusable, easy to wash and prepare for the next truffle. It is possible to have several amulets, depending upon, for instance, whether there is a difference between the night cosmetic and the day cream as well as perhaps more than one amulet for different treatment formulations. All amulets are capable of attaching to the same motor platform.

Accordingly, a new cosmetic system has been described using a cosmetic truffle member, which is individually made and then processed by the user in an amulet appliance to blend and heat the truffle, producing a warm, fragrant cream for application to the user's face, body or hair.

Although a preferred embodiment of the invention has been disclosed for purposes of illustration, it should be understood that various changes, modifications and substitutions may be incorporated in the embodiment without departing from the spirit of the invention, which is defined by the claims which follow.

1. A method for preparing a skin formulation, the method comprising:
placing a cosmetic article into an appliance configured to receive, heat, and blend the cosmetic article,
wherein the appliance comprises a container configured to receive the cosmetic article, a heating assembly configured to heat the cosmetic article when the cosmetic article is received within the container, and blending elements configured to blend the cosmetic article into the skin formulation; and
wherein the placing comprises placing the cosmetic article into the container of the appliance;
wherein the cosmetic article comprises:
a water resistant outer shell having a melting point in the range of 90-110 degrees Fahrenheit, a thickness of 0.5 mm-5 mm, and that defines an interior volume;
a cosmetic material contained within the interior volume of the outer shell; wherein the cosmetic material and the outer shell are configured to blend with one another to produce the skin formulation; and
heating and blending the cosmetic article in the container of the appliance to form the skin formulation, wherein the blending comprises moving the blending elements to blend the cosmetic article within the container, and wherein the heating comprises heating the cosmetic article within the container with the heating assembly.

2. The method of claim 1, wherein the skin formulation is formed entirely from the cosmetic article.

3. The method of claim 1, wherein the outer shell of the cosmetic article comprises an oil and wax mixture.

4. The method of claim 1, wherein the interior volume of the outer shell of the cosmetic article has a volume in the range of 0.5 ml to 2 ml or a volume that is 4 ml or larger.

5. The method of claim 1, wherein the thickness of the outer shell of the cosmetic article is in the range of 3.1 mm to 5 mm.

6. The method of claim 1, wherein the outer shell of the cosmetic article further comprises a resin selected to increase the melting point of the outer shell.

7. The method of claim 1, wherein the outer shell of the cosmetic article is configured to melt at temperatures in the range of 105-110 degrees F.

8. The method of claim 1, wherein the cosmetic material comprises a cosmetic that is configured to one or more of cleanse, hydrate, treat, protect, and color skin.

9. The method of claim 1, wherein the cosmetic material comprises an oil-based formula.

10. The method of claim 1, wherein the cosmetic material comprises a water-based formula.

11. The method of claim 1, wherein the cosmetic article further comprises an active ingredient that is configured to treat one or more skin care issues, and further wherein the one or more skin care issues include one or more of aging and acne.

12. The method of claim 11, wherein the active ingredient comprises retinol.

13. The method of claim 1, wherein the cosmetic article further comprises an active ingredient that is configured to treat one or more skin care issues, and further wherein the active ingredient includes one or more of a prescription ingredient, vitamin A, vitamin E, vitamin C, beta-hydroxy acid, salicylic acid, alphahydroxy acid, a protein, a peptide, a skin darkening agent, a skin lightening agent, and a liposome.

14. The method of claim 1, wherein the cosmetic article includes an active ingredient contained within a capsule within the interior volume of the outer shell.

15. The method of claim 14, wherein the capsule comprises an outer coating that isolates the active ingredient from the cosmetic material when the active ingredient and the cosmetic material are within the interior volume of the outer shell.

16. The method of claim 1, wherein the cosmetic article is configured such that hand action alone is insufficient to heat and blend together the outer shell and the cosmetic material to form the skin formulation.

17. The method of claim 1, wherein the appliance further comprises a motor and a drive member configured to drive the rotation of the blending elements to blend the cosmetic article, and further wherein the heating assembly is operable to heat the cosmetic article as the blending elements blend the cosmetic article to produce the skin formulation.

18. The method of claim 1, wherein the method further comprises mashing the cosmetic article in the container prior to blending the cosmetic article to form the skin formulation.

19. The method of claim 18, wherein the appliance comprises a top portion and a bottom portion that together define the container, wherein the method further comprises mashing the cosmetic article against the blending elements.

20. The method of claim 1, wherein the container is spherical, wherein the container includes a top portion and a bottom portion, and wherein the top portion is movably connected to the bottom portion.

21. The method of claim 1, wherein the blending elements include curved blades.

22. The method of claim 1, wherein the heating and blending comprises providing electrical current to the heating assembly, wherein the appliance comprises a motor configured to move the blending elements, wherein the heating and blending comprises providing electrical current to the motor to move the blending elements, wherein the appliance further comprises an on/off button configured to control provision of electrical current to the heating assembly and the motor, and wherein the method further comprises actuating the on/off button to provide electrical current to the motor and the heating assembly.
